(19) United States
(12) Patent Application Publication
(10) Pub. No.: US 2013/0053677 A1
(43) Pub. Date: Feb. 28, 2013

(54) SYSTEM AND METHOD FOR WOUND CARE MANAGEMENT BASED ON A THREE DIMENSIONAL IMAGE OF A FOOT

(71) Applicant: Jeffrey E. Schoenfeld, Naples, FL (US)

(72) Inventor: Jeffrey E. Schoenfeld, Naples, FL (US)

(21) Appl. No.: 13/598,434

(22) Filed: Aug. 29, 2012

Related U.S. Application Data
(63) Continuation-in-part of application No. 12/941,478, filed on Nov. 8, 2010.
(60) Provisional application No. 61/259,384, filed on Nov. 9, 2009.

(51) Int. Cl. A61B 5/107; A61B 6/00

(52) U.S. Cl. 600/407

(57) ABSTRACT

Disclosed is a system for wound care management of the diabetic foot. In an embodiment, the system uses a scanner configured to obtain a two-dimensional image of the plantar surface of the foot. This system also includes a wound measurement tool to measure the diameter and volume of the diabetic ulcer. Additionally, this system includes an image processor that converts a two-dimensional image of the diabetic ulcer into a three-dimensional map. The system also includes a measurement calculator, using the three-dimensional map, that accurately measures the diameter and volume of each ulcer on the plantar of the foot. All scans are stored indefinitely and can be compared in a side-by-side setting, at the same time analyzing and comparing progress of the ulcer treatment in a wireframe mode. Other embodiments are also disclosed.

### REFERENCE TO PENDING PRIOR PATENT APPLICATION

This patent application is a continuation-in-part of pending prior U.S. patent application Ser. No. 12/941,478, filed Nov. 8, 2010 by Jeffrey E. Schoenfeld for SYSTEM AND METHOD FOR DESIGNING AN INSERT BASED ON A THREE DIMENSIONAL IMAGE OF A FOOT, which in turn claims the benefit under 35 U.S.C. 119 (e) of U.S. Provisional Patent Application No. 61/259,384, filed Nov. 9, 2009 by Jeffrey E. Schoenfeld for SYSTEM AND METHOD FOR DESIGNING AN INSERT BASED ON A THREE DIMENSIONAL IMAGE OF A FOOT. The above-identified patent applications are hereby incorporated herein by reference.

### BACKGROUND

1. Field of the Invention

This invention relates to the field of digital shape acquisition and foot wound management.

2. Background

In an effort to address patients' needs, physicians are advancing their practices with blogs, text messaging, and portable electronic tablets. Blogs help physicians with educating patents and fellow practitioners. Text messaging may be used with appointment reminders and issues to ensure legal compliance. Portable electronic tablets may be implemented to expedite documentation.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In an embodiment, there is provided a system for managing diabetic ulcers on a foot, comprising a scanner configured to obtain a two-dimensional image of a plantar surface of a foot; an ulcer wound tracer tool configured to enclose a diabetic ulcer wound illustrated in the two-dimensional image separate from another portion of the surface of the foot; an image processor configured to convert the two-dimensional image of the diabetic ulcer wound enclosed by the wound marking tool separate from the another portion of the surface of the foot via software into a three-dimensional map; and a measurement calculator, using the three-dimensional map, configured to determine at least one of a surface area of the diabetic ulcer wound along the surface of the foot and a volume of the wound below the surface of the foot.

In another embodiment, there is provided a method of wound care management for a foot, comprising scanning, with a scanner, a two-dimensional image of the surface of a foot; marking, using an ulcer wound tracer tool, to enclose an ulcer wound illustrated in the two-dimensional image separate from another portion of the surface of the foot; converting, using an image processor, the two-dimensional image of the ulcer wound enclosed by the ulcer wound tracer tool separate from the another portion of the surface of the foot into a three-dimensional map; and measuring, using the three-dimensional map, to determine at least one of a surface area of the diabetic ulcer wound along the surface of the foot and a volume of the diabetic ulcer wound below the surface of the foot.

Other embodiments are also disclosed.

These and other systems, methods, objects, features, and advantages of the present invention will be apparent to those skilled in the art from the following detailed description of the preferred embodiment and the drawings. All documents mentioned herein are hereby incorporated in their entirety by reference.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

The invention and the following detailed description of certain embodiments thereof may be understood by reference to the following figures:

FIG. 1 depicts a system for designing a foot support device;

FIG. 2 depicts a logical flow of a method for designing a foot support device;

FIG. 3A depicts a foot scanner for capturing a two-dimensional image of the surface of a foot;

FIG. 3B depicts the foot scanner with a foot disposed on the scanning surface;

FIG. 4 is a graphical user interface (GUI) for a software system used with the foot scanner;

FIGS. 5 and 6 are illustrations of a two-dimensional scan of a foot from the foot scanner;

FIGS. 7 and 8 illustrate three-dimensional images rendered from the scan depicted in FIG. 5;

FIG. 9 is a GUI for a software system for a set of left foot and right foot scans for a patient;

FIG. 10 is a GUI for a software system used with the foot scanner;

FIG. 11 is a GUI for patient education;

FIG. 12 is a GUI for measurement and tracking of foot wounds;

FIG. 13 is a GUI for comparing scans of feet;

FIG. 14 is a two-dimensional foot scan image of a plantar surface of a foot having a wound;

FIG. 15 is a GUI for measurement of a wound within the image of FIG. 14 and provides a marking tool to mark the wound surface;

FIG. 16 is a three-dimensional rendering of the wound and is created from the marked region containing the wound in the two-dimensional image of FIG. 15;

FIG. 17 is another view of the three-dimensional rendering of the wound of FIG. 16;

FIG. 18 is a wireframe image of the wound illustrated in FIGS. 15-17;

FIG. 19 is a GUI illustrating a management system for managing patient scans;

FIG. 20 is a side-by-side comparison of scans of a foot in which the wound has healed over time and is smaller in the right side image; and

FIG. 21 is a GUI illustrating the wounds of FIG. 20 in wireframe views together with area and volume measurements.

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

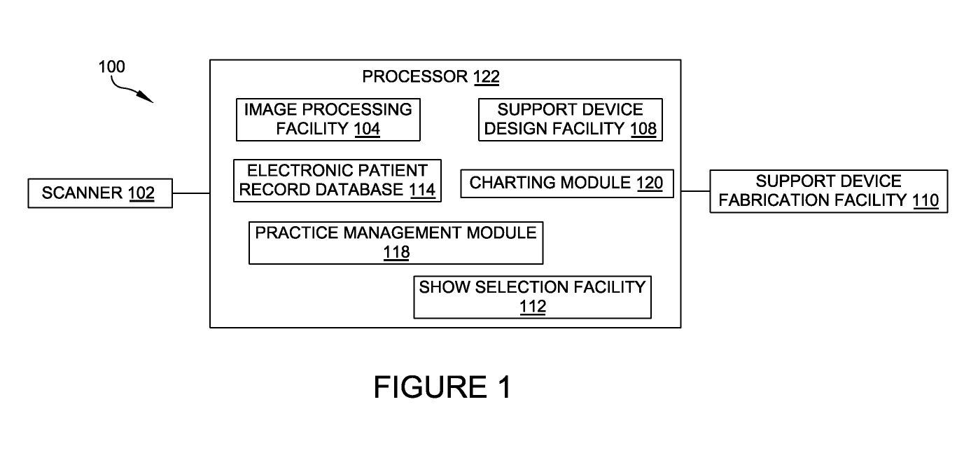

Referring to FIG. 1, a shape acquisition system 100 for designing a support device for a foot may include a scanner 102 for obtaining a two-dimensional image of the plantar surface of a foot, an image processing facility 104 for converting the two-dimensional image of the surface of the foot into a three-dimensional map, and a support device design facility 108 for designing a support device based on the foot parameters from the three-dimensional map. The support device design facility 108 may be adapted to modify the support device design based at least in part on a foot abnormality. The image processing facility 104 and support device design facility 108 may be embodied as software or applications stored on a processor 122 or server associated with the scanner 102.

The shape acquisition system 100 may capture more natural foot shape models in a non-weight bearing format, resulting in a truer foot image and a better orthotic fit. The user may place their left foot, right foot, or both feet on the scanner 102 for obtaining a two-dimensional image of the plantar surface of the foot. A foot support on the scanner 102 may enable non-weight bearing scans of the foot. The scanner may acquire an image of the foot in gray-scale, color, black-and-white, and the like. The scanner may optionally be fitted with a larger scanning surface to accommodate larger feet.

The shape acquisition system 100 may also comprise an LCD thermometer as well as pressure sensing devices, which may facilitate diabetic care.

The shape acquisition system 100 may comprise parallel phased array computing, where processors are slaved together and adapted to iteratively process an input shape to determine a match from among a shape library. In embodiments, any body part shape possible thus eliminating the need for costly casting. In embodiments, no human intervention to find a shape match may be necessary.

An image processing facility 104 may convert the two-dimensional image of the surface of the foot into a three-dimensional model of the foot. The image processing facility 104 may obtain the three-dimensional map by measuring the color and/or intensity of a pixel of the two-dimensional image and assigning the pixel a distance from the scanner based on its color and/or intensity. For example, the two-dimensional image may be monochromatic and each pixel may correspond to a shade of gray along a gray-scale. In another embodiment, the two-dimensional image may be polychromatic and each pixel may correspond to a color. Each shade of gray or each color may correspond to a particular distance from the surface of the scanner. The image processing facility 104 may process each pixel in the image by assigning each pixel a distance from the scanner, The correspondence between color/shade of gray and distance from the scanner may be empirically derived. Once the pixels in the image are processed, the distances obtained may be used to construct a three-dimensional map of the imaged surface of the foot. The maps may be rotated in 360 degrees, zoomed, displayed in full screen, and displayed in at least one of wire frame, solid, textured, surface and topographical views on a graphical user interface of the image processing facility. The map may be magnified to enable the viewing of a particular foot abnormality or pathology. Image processing may be enabled by CAD/CAM technology.

Foot parameters may be derived from the three-dimensional map. For example, at least one of the height, length, curvature, and position of the arch may be determined from the three-dimensional map. In another example, at least one of the width, curvature, shape, and size of the heel may be determined from the three-dimensional map. A support device design facility 108 may be used to design a support device based on the foot parameters from the three-dimensional map, such as arch height, heel shape and heel size. Other parameters may also be used in the design of the support device, such as foot size, width, user weight, user gender, age, health concerns, and the like. The support device design facility 108 may generate a custom design for a support device based on at least one parameter.

The support device design facility 108 may be adapted to modify the support device design based at least in part on a foot abnormality. For example, a diabetic user may have a sore on a plantar surface of her foot. The dimensions and position of the sore may be determined from the three-dimensional map. In the example, the support device may be designed with a void at the position of the sore. Other modifications to the support device due to other foot abnormalities or pathologies are contemplated and are encompassed herein.

The shape acquisition system 100 may comprise an electronic patient record database 114 for storing the two-dimensional image and the three-dimensional model in association with patient demographics and the custom design. The electronic patient record database 114 allows users to maintain an ongoing log of patient scans, tracking progress throughout the treatment process. The scans may be saved for later use or printed, optionally with patient information.

The shape acquisition system 100 may provide patient education based on the scan and any abnormalities present on the scan or conditions known to exist, either based on the scan or otherwise indicated. For example, patient education may relate to arch pain, calluses, arthritis, diabetes, heel pain, metatarsalgia, plantar fasciitis, ankle sprains, shin splints, bunions, neuromas, leg length discrepancy, and the like. The shape acquisition system 100 may have the ability to customize the education to the patient or doctor's practice, print the patient education, view in various formats, and the like. For example, the education may include patient education notes and treatment instructions. Patient education may include treatment algorithms. For example, patient education may include animated visuals for the diabetic foot, such as 3D-RX visuals, FLASH animation visuals, HTML visuals, and the like. Patient education may be embodied in video, audio, animation, text, and the like.

In embodiments, the shape acquisition system 100 may be a centerpiece of a multi-lingual education and treatment e-center. In an embodiment, a doctor may mark a diagnosis or course of treatment on the user interface and relevant applications or education modules may be identified based on the diagnosis/treatment.

The user interface may include applications directed at diabetes products, diabetes services, diabetes patient education, and the like.

The shape acquisition system 100 may include a system for engaging in a referral network. For example, once a patient has been scanned and a diagnosis is entered into the system 100, the user may search a referral network to identify a provider for continued care. The search may begin automatically when the diagnosis is entered.

The shape acquisition system 100 may be embodied as a mobile cart, a portable model, a scanner and computer combination such as with a tablet PC, laptop, desktop computer, and the like. The system 100 may operate wirelessly, such as to update a database 114, automatically detect and transparently install any necessary software updates, wirelessly transmit diagnosis, treatment information, scans, etc. to a patient records facility or a milling facility, and the like.

The design may be ordered as a support device by uploading the design as an electronic order to a support device fabrication facility 110 for fabrication of the support device based on the support device design generated by the support device design facility 108. Fabrication of the support device may commence from a support device template or may commence de novo from starting materials. The user may have the ability to add notes to each product ordered, place an order on hold, select multiple products, change product options after selecting the product, view orders in a shopping cart environment, delete orders before finalizing orders, view each product ordered and print details, place the order on rush, ship the order directly to the patient, ship the order to an alternate address, rush the shipment, display a number of items in a shopping cart, display a quick reference of items ordered on the main screen, edit order after submission, and the like. The system 100 may include a “Favorites” or Preset Button for default orders. The electronic order may include information regarding the patient's diagnosis. A facility may enable converting a patient summary screen into PDF for patient records.

The design may also be uploaded to a shoe selection facility 112 for selecting a shoe that can accommodate the support device.

The shape acquisition system 100 may include a practice management module 118. The practice management module 118 may further include a scheduling module, an e-claims module, an insurance verification facility, and the like.

The shape acquisition system 100 may include a charting module 120. The charting module 120 may enable a user to take a patient history, create pressure mapping tracking/graphs, create temperature sensor tracking/graphs, and the like.

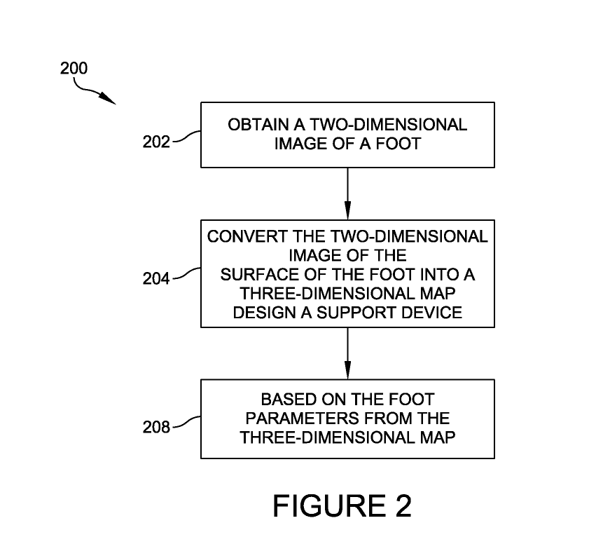

Referring to FIG. 2, a method for designing a support device for a foot may include obtaining a two-dimensional image of the surface of a foot 202; converting the two-dimensional image of the surface of the foot into a three-dimensional map 204; and designing a support device based on the foot parameters from the three-dimensional map 208. The design may be at least partially based on a foot abnormality. The design may be at least partially based on a foot abnormality. Converting the two-dimensional image to a three-dimensional map may include measuring the color and/or intensity of a pixel of the two-dimensional image and assigning the pixel a distance from the scanner based on its color and/or intensity. The pixel may be a color or gray-scale pixel. The method may further include fabricating the support device based on the support device design.

In an embodiment, the shape acquisition system 100 may also be integrated with a patient administration system, patient management technology, patient retention technology, patient communication technology, and a digital patient records facility.

The methods and systems described herein may be deployed in part or in whole through a machine that executes computer software, program codes, and/or instructions on a processor. The processor may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements.

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, programs codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer to peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps thereof, may be realized in hardware, software or any combination of hardware and software suitable for a particular application. The hardware may include a general purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

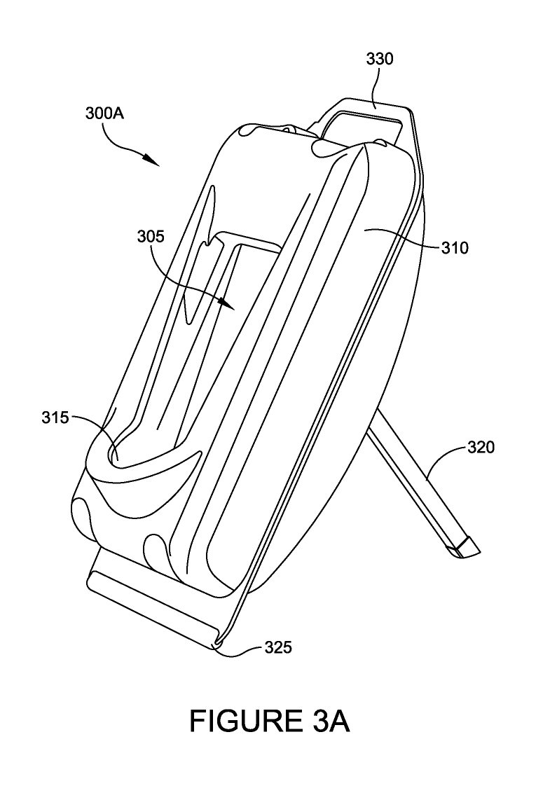

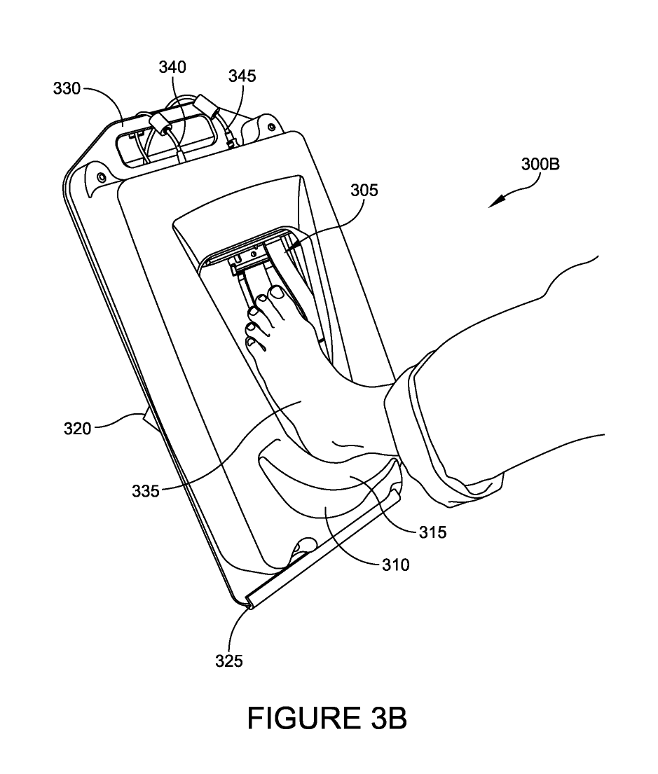

With reference to FIGS. 3A and 3B, and in an embodiment, there is provided a portable, integrated foot scanner unit 300A/300B. Scanner 300A/300B delivers a cost-effective solution providing advances in diagnosis, treatment and care for the foot. Scanner 300A/300B and the related software and methods disclosed herein are especially suitable for the diabetic patient.

Still referring to FIG. 3A, there is shown an exemplary embodiment of foot scanner 300A. A glass portion scanning surface 305 is disposed within a housing 310 and may include a heal support 315. A support 320 and a support 325 are provided to dispose glass portion 305 at an angle to prevent the foot from providing a distorted image through significant patient weight bearing, or weight transfer, onto the glass. A handle 330 may be provided for transporting or repositioning scanner 300A/300B (FIG. 3B.)

With reference now to FIG. 3B, foot scanner 300B is shown with a foot 335 disposed on the scanning surface 305. In addition, communication cables 340 and 345 are shown in operable connection with scanner 300B.

In one embodiment, the conversion of a 2D image from a white light flatbed scanner is accomplished by the following tasks. The image is scanned using the flatbed scanner and converted to a gray scale picture. This picture is then reduced in scale. In an embodiment, this reduction may be 15% of the original size on machines with more than 1 GB of video RAM and 8% of the original size on machines with less video RAM than that amount. The gray values are then converted to distance measurements using a table of pre-calculated values which have been tested and verified on scanners. These distance values are then used to calculate a height map which is then constructed using vertices which make up a 3D image.

This 3D image may be displayed using Microsoft DirectX technology and, once built, a texture may be applied (the texture is obtained from the original 2D scan). The edges are trimmed to provide a more presentable picture without jagged edges. The remaining points in this 3D image are then gathered to create a Stereo Lithography file. This “STL” file is then saved in binary format and transmitted to a central server.

The system provides enhanced patient educational materials improving patient compliance and speeding up the healing process. The system utilizes the same graphics engine as Xbox 360®, boasting 150 points of measurement per square inch. Corrective and offloading devices are now prescribed with the accuracy of plaster casting without the mess. The system of its kind to produce a 1-to-1 image of the foot. Patients have never been provided a mirrored image of the foot in such clarity. Patients make referrals to family and friends.

Educating patients with the materials from the system increases patient compliance and helps to prevent future complications. The system is the tool for practitioners to provide meticulous attention to foot care and proper management of foot injuries.

With the ability to provide an unlimited amount of stored scans, the system places wound images side by side to show the healing process. Additionally, as requirements for Medicare and private insurance reimbursement are continuously more difficult, storage of scans provides significant advantages. Practitioners can be sure they have all appropriate documentation, which will never be lost. The 150 points of measurement per square inch provide unmatched accuracy.

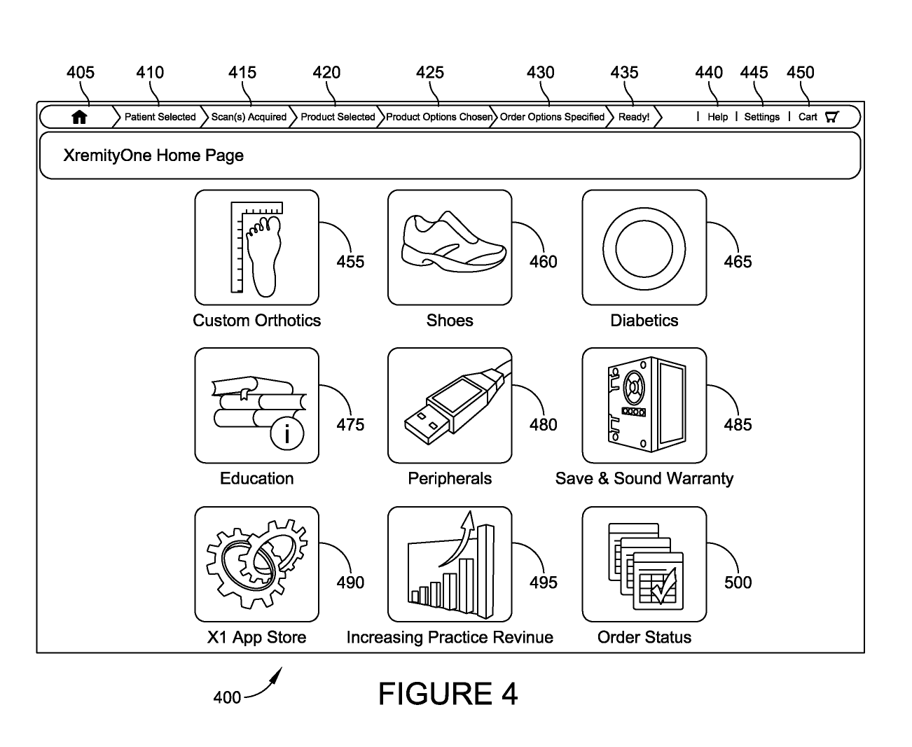

Together with a laptop computer, or other computer device, imaging software, the system serves as a single portal whereby practitioners are able to scan patients' feet, store unlimited patient information and complete the process with immediate order submission of orthotics, diabetic inserts and/or diabetic shoes. FIG. 4 illustrates a home page with various icons for invoking various aspects of the system. In the graphical user interface (GUI) 400 of FIG. 4, there is provided a home button 405 to return the user to this page of the GUI 400. Button 410 allows selection of a patient. Button 415 allows selection of the scans acquired by the system. Button 420 allows selection of a product. Button 425 allows selection of product options. Button 430 allows ordering of specified options. Button 435 allows review of an order. Button 440 invokes a help process. Button 445 invoices a settings section. Button 450 allows review of an order cart. Button 455 provides a module to order custom orthotics. Button 460 provides a module to order shoes based on the foot scan. Button 465 provides a module related to diabetes features. Button 475 provides a module for patent education. Button 480 provides a module for integration of various peripherals for use with the system. Button 485 provides access to a section on warranty information. Button 490 provides access to an online “app” ordering store. Button 495 provides access to a module providing information on practice revenue related to the system. Button 500 provides information related to order status.

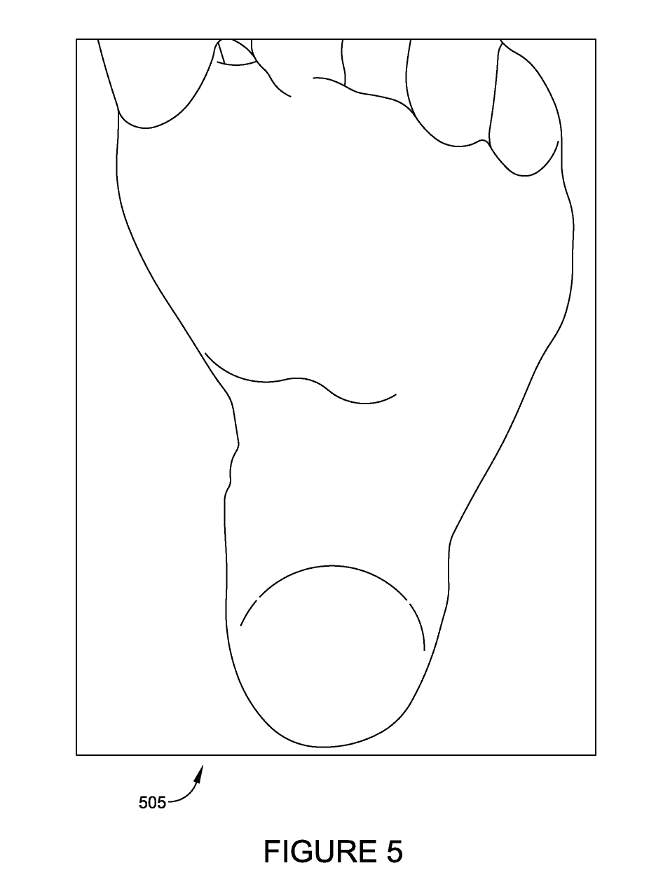

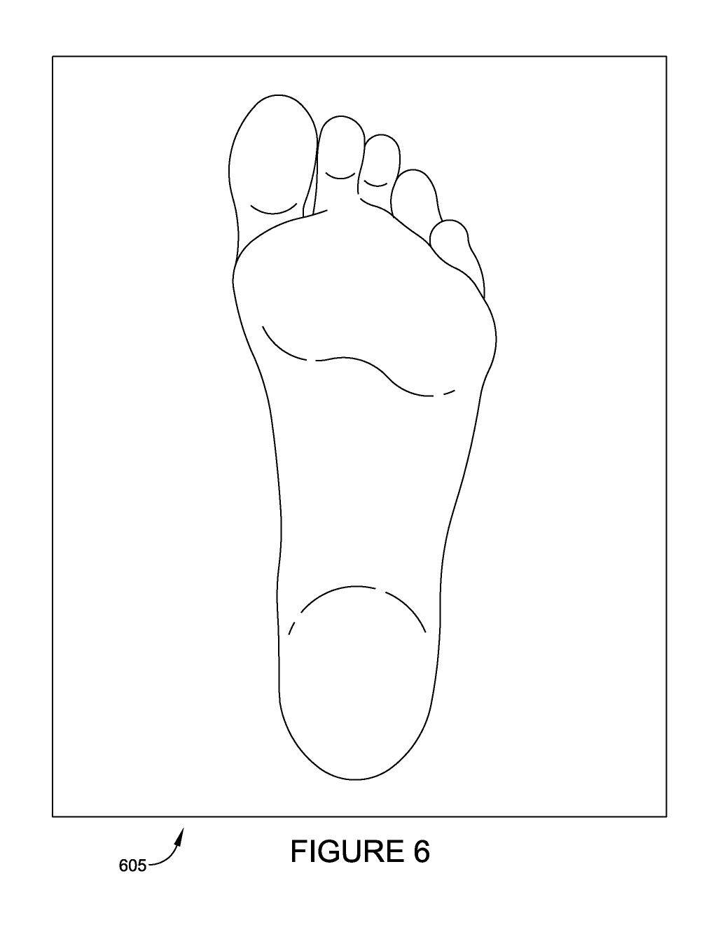

With reference to FIGS. 5 and 6, are illustrations of a two-dimensional scan 505/605 of a foot from the foot scanner 300A/300B. The system features Xbox 360® graphics that capture an image of the foot, then creates a 3D model in seconds.

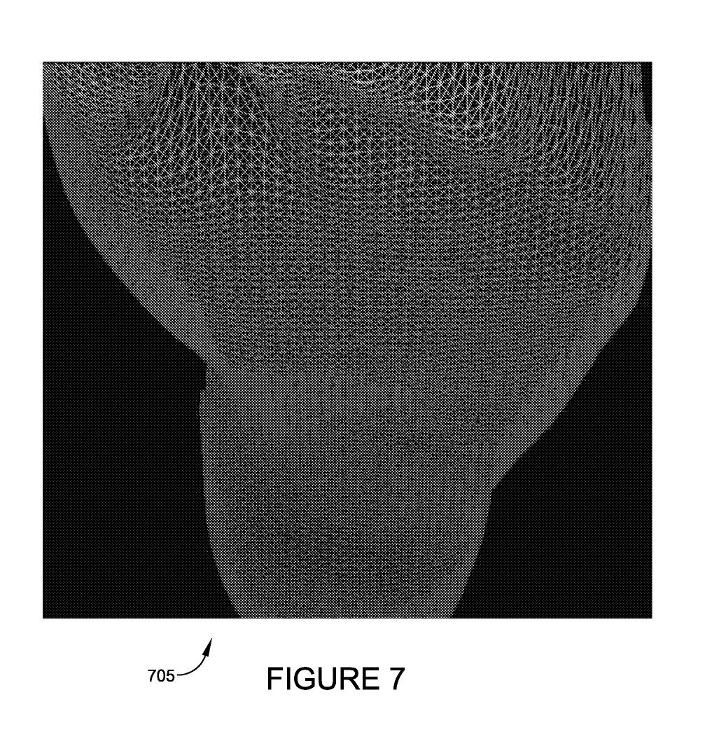

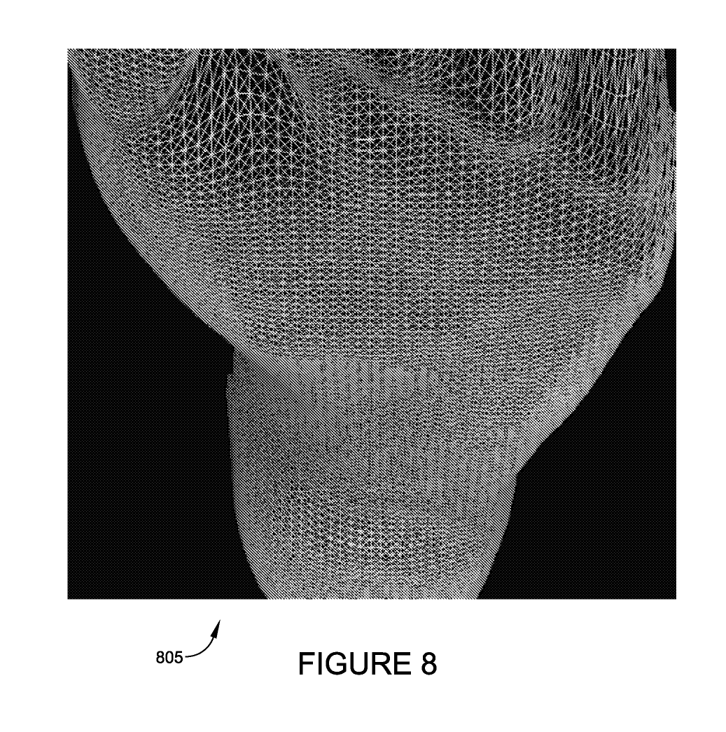

With reference to FIGS. 7 and 8, there are shown three-dimensional images 705 and 805 rendered from the scan depicted in FIG. 5. The precision of the scan gives 150 points of measurement per square inch ensuring a more accurate diagnosis, fitting and treatment. FIG. 7 illustrates a topical wireframe illustration of a scan of a foot with 150 points (shown as intersections) per square inch. FIG. 8 is a textured wireframe showing 150 points (shown as intersections) per square inch. Patients are seeing a mirrored, 1-to-1 image of his or her feet. This illustration exponentially increases the effectiveness of patient documentation.

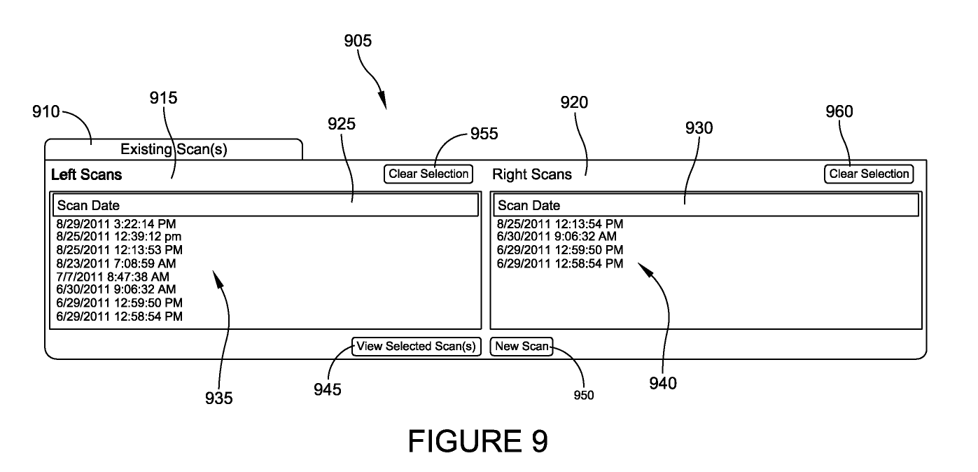

With respect to FIG. 9, a GUI 905 may be provided for a software system for storing a set of existing scans 910. These may include a set of left foot scans 915 and right foot scans 920 for a patient. The system is capable of holding an unlimited number of images. This is particularly salient in wound healing as the storage of scanned images allows podiatrists to more accurately document a wound's healing progress. GUI 905 may also include various fields including, for example, left foot scan date 925, right foot scan date 930, left foot scan information 935, right foot scan information 940, a view button 945 to view selected scans, and a new scan button 950.

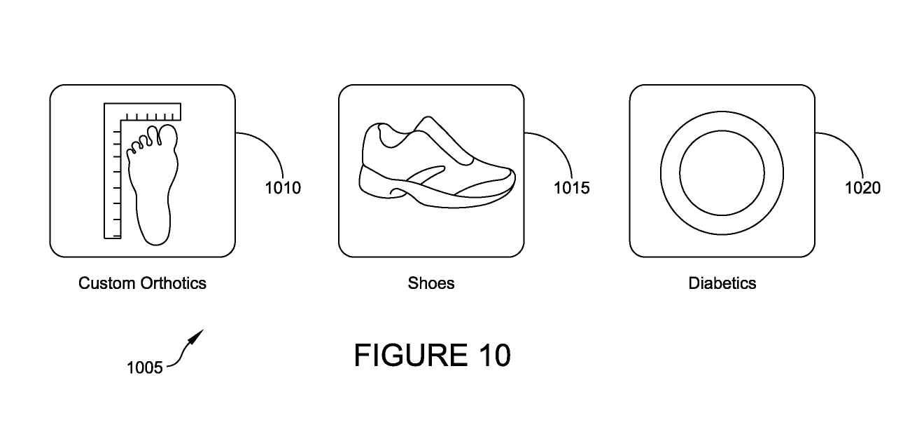

In an embodiment, the system may resemble an iPod or smart phone “app” format, giving users a multitude of system functions. With reference to FIG. 10, and in an embodiment, once a patient's foot is scanned, a series of diagnosis-specific products appear based on the data garnered from the scan. This GUI 1005 may provide various products including, for example, custom orthotics 1010, shoes 1015, and diabetes software 1020.

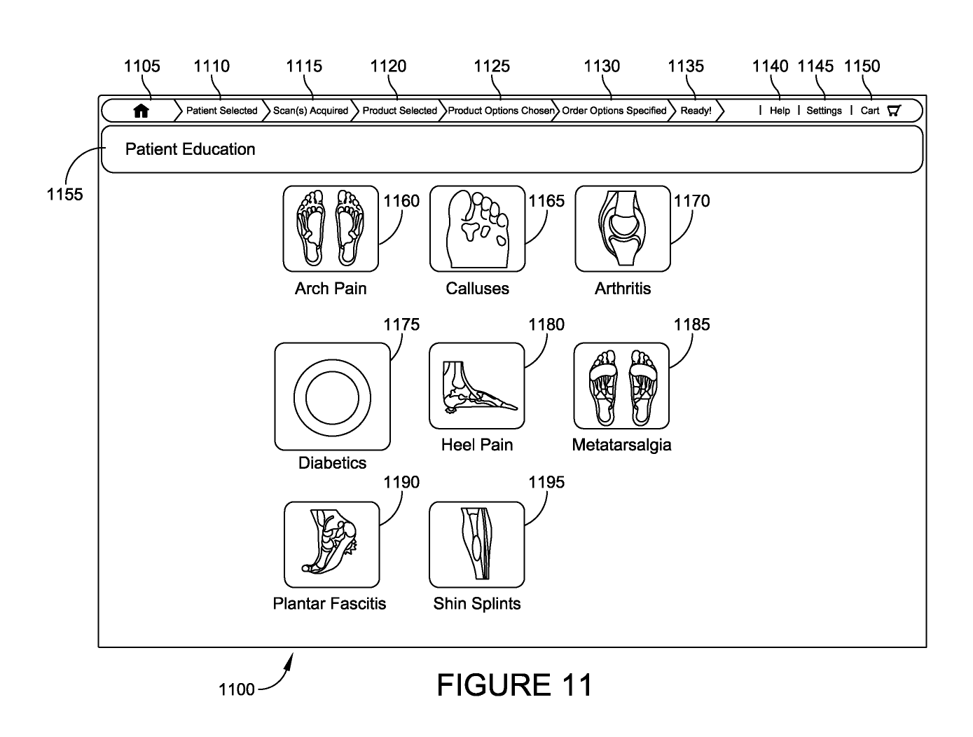

With reference to FIG. 11, a GUI 1100 may be provided for patient education. Not only do the detailed graphics help patients to visualize their specific foot pathologies, but the system is continuously building upon its educational elements. Improved patient education will increase their “doctor's orders” compliance, expediting healing. GUI 1100 may include a home button 1105, a patient selected button 1100, a scan acquired button 1115, a product selected button 1120, a product options chosen button 1125, an order options specified button 1130, a ready button 1135, a help button 1140, a settings button 1145, and a cart button 1150. A banner 1155 may specify the components offered by GUI 1100. Various modules may also be provided, which may include, for example, an arch pain module 1160, a calluses module 1165, an arthritis module 1170, a diabetes module 1175, a heel pain module 1180, a metatarsalgia module 1185, a planar fasciitis module 1190, and a shin splints module 1195.

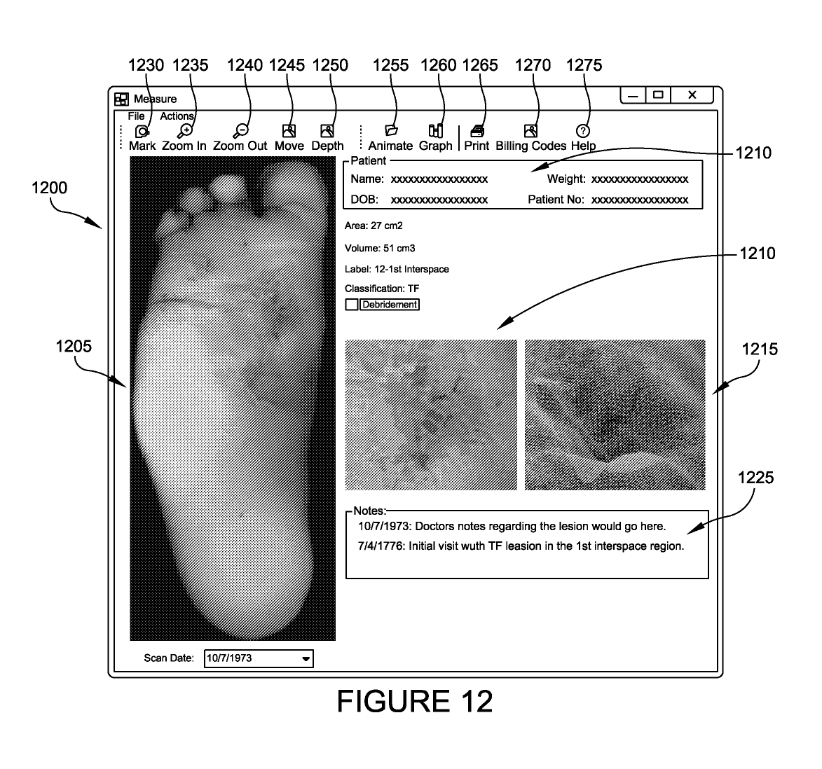

Referring to FIG. 12, there is illustrated a graphical user interface (GUI) 1200 for measurement and tracking of foot wounds with a portion 1205 showing a plantar view of the foot with a wound. Another portion of GUI 1200 illustrates an enlarged image 1210 of the wound. A wireframe 1215 portion illustrates the wound in another portion of GUI 1200. A patent data section 1220 may provide patient specific information display. A notes section 1225 may provide a physician notes display. Various buttons may be provided, which may include, but are not limited to, a mark button 1240, a zoom in button 1235, a zoom out button 1240, a move button 1245, a depth button 1250, an animate button 1255, a graph button 1260, a print button 1265, a billing codes button 1270, and a help button 1275.

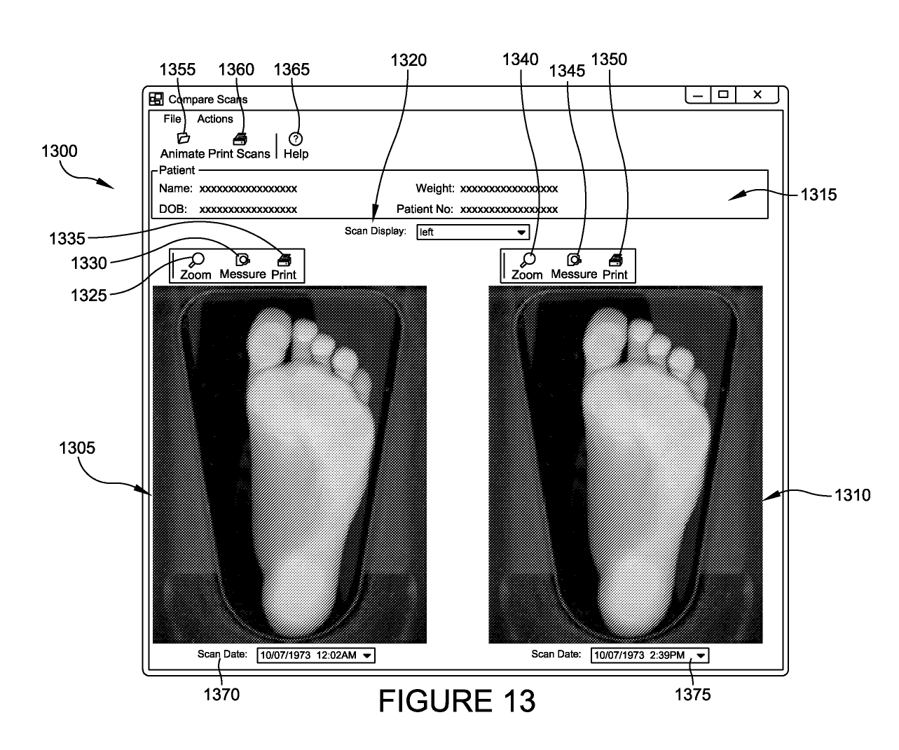

FIG. 13 illustrates a GUI 1300 with dual images 1305 and 1310 to compare scans. In the left-hand portion, an earlier scan 1305 is illustrated. In the right-hand portion, a later scan 1310 is illustrated. This allows side-by-side comparison of scans. Patient information may be provided in a display portion 1315. A selector 1320 may be provided to select a scan for display. Various buttons may be provided, including, a zoom button 1325, a measure button 1330, a print button 1335, a zoom button 1340, a measure button 1345, a print button 1350, an animate button 1355, a print scans button 1360, and a help button 1365. Scan date selectors 1370 and 1375 may also be provided to easily select images from various dates.

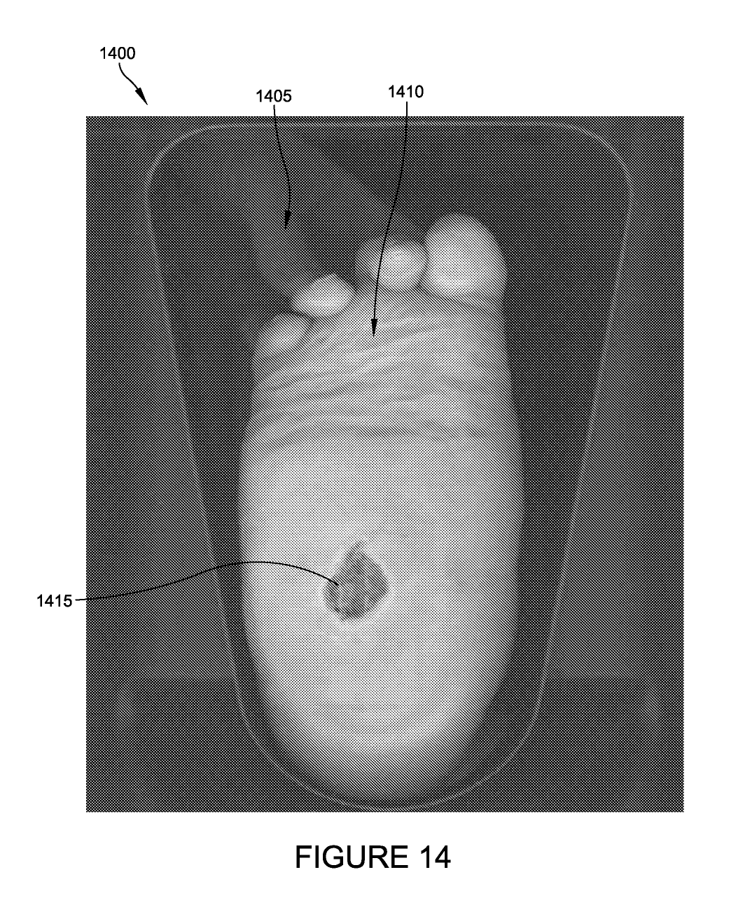

Referring to FIG. 14, there is shown a two-dimensional foot scan image 1400 within a scanning plane 1405 for a plantar surface of a foot 1410 having a wound 1415. This particular image is of the right foot of a patient having an ulcer as a foot wound.

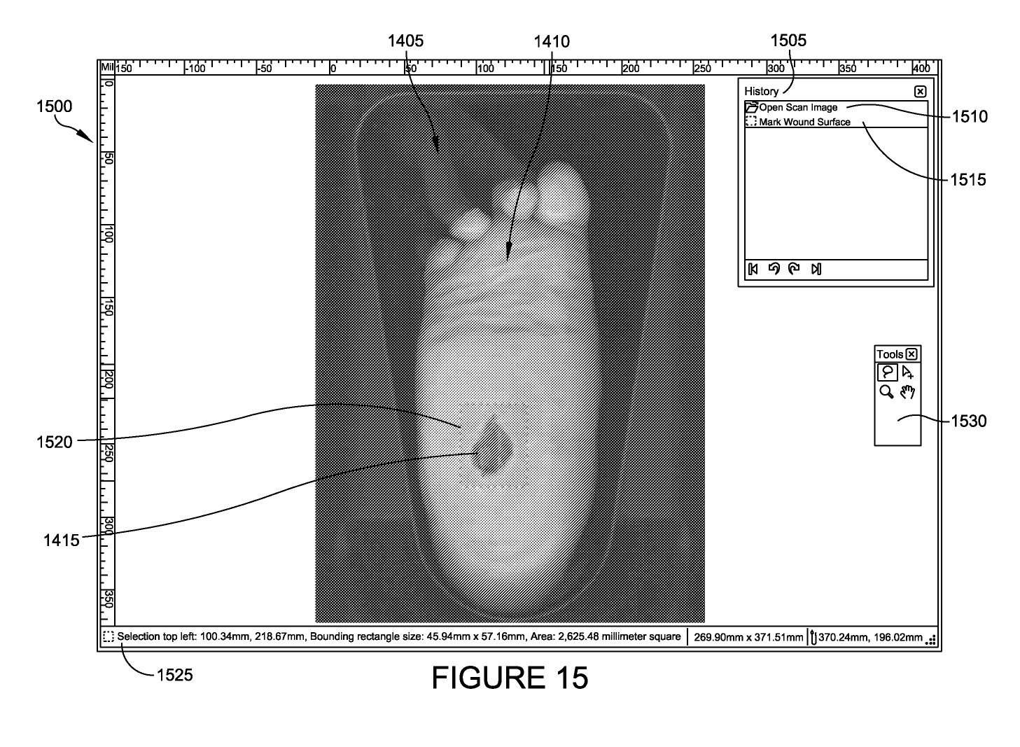

In FIG. 15, there is shown a GUI 1500 for measurement of wound 1415. A button 1510 is provided to open a scan image. A mark wound surface button 1515 provides a marking tool 1520 to mark a perimeter of the wound surface. A measurement tool 1525 calculates and displays various attributes about the marked wound. Other tools 1530 may be provided for analysis of the wound 1415 as the marking tool is used to mark the wound surface for analysis.

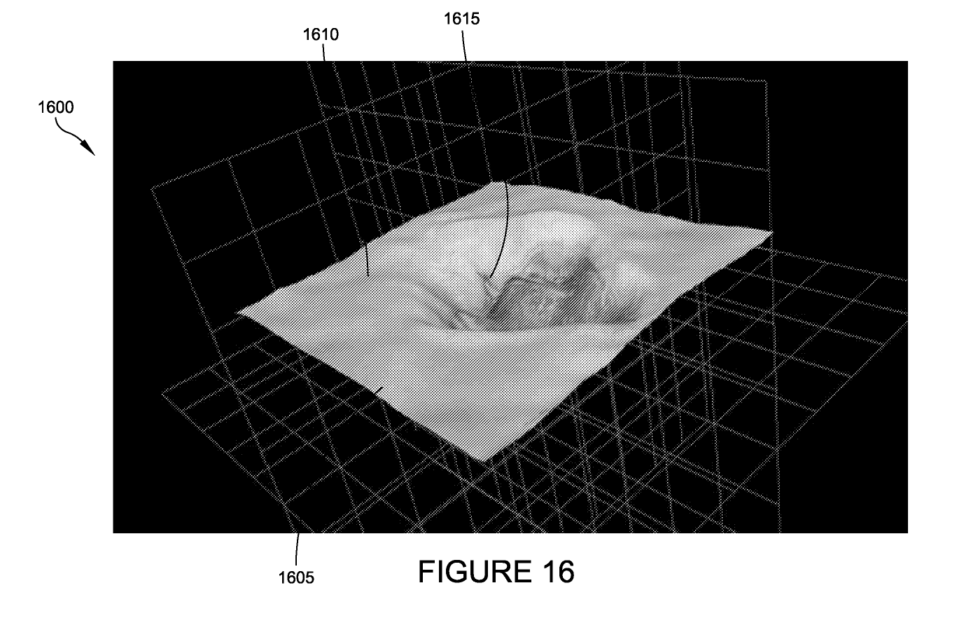

In FIG. 16, there is shown a three-dimensional image 1600 of a selected portion 1605 of wound surface 1610, which was previously marked for analysis. Wound 1615 is shown within selected portion 1605.

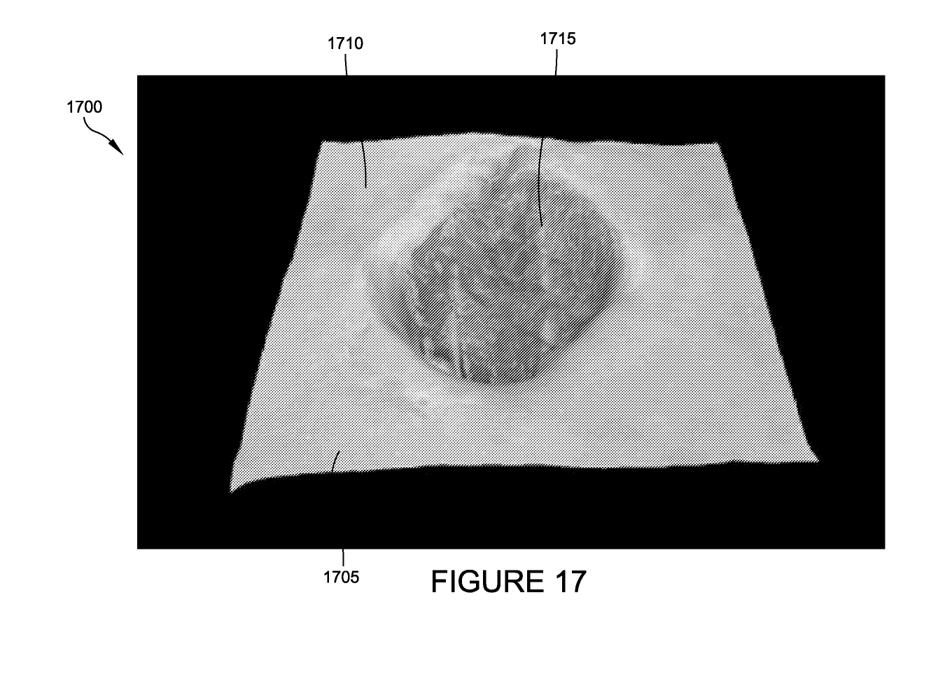

FIG. 17 illustrates a rotated view of image 1600 showing a more planar image 1700 with a selected portion 1705 of wound surface 1710 and containing wound 1715.

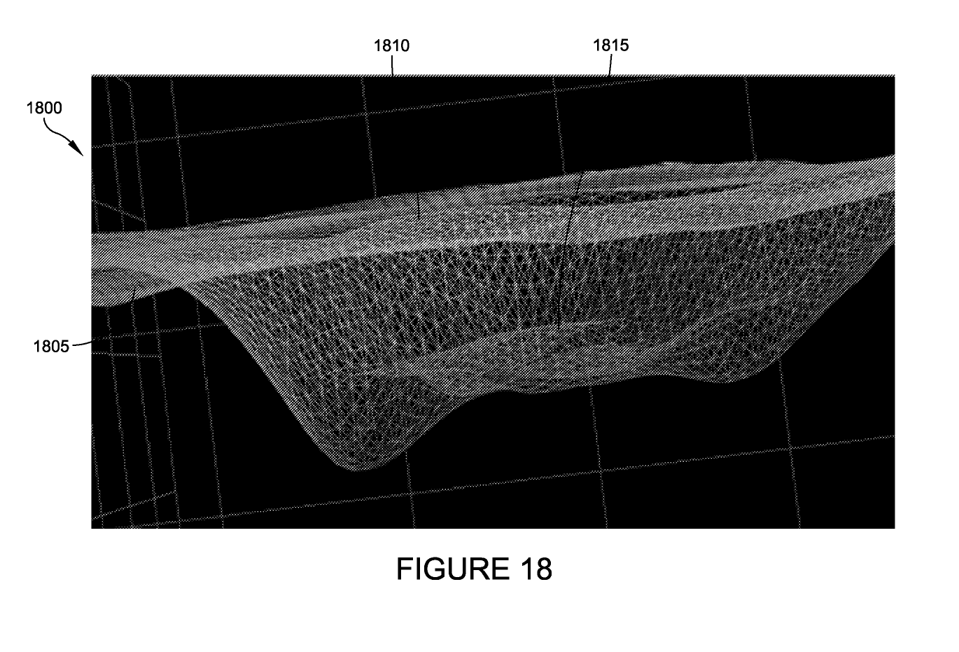

FIG. 18 illustrates a wireframe view 1800 of wound 1515. View 1800 includes a selected portion 1805 with a wound surface 1810 together with the a contoured wound depth wireframe 1815 as calculated from the wound surface previously marked for analysis.

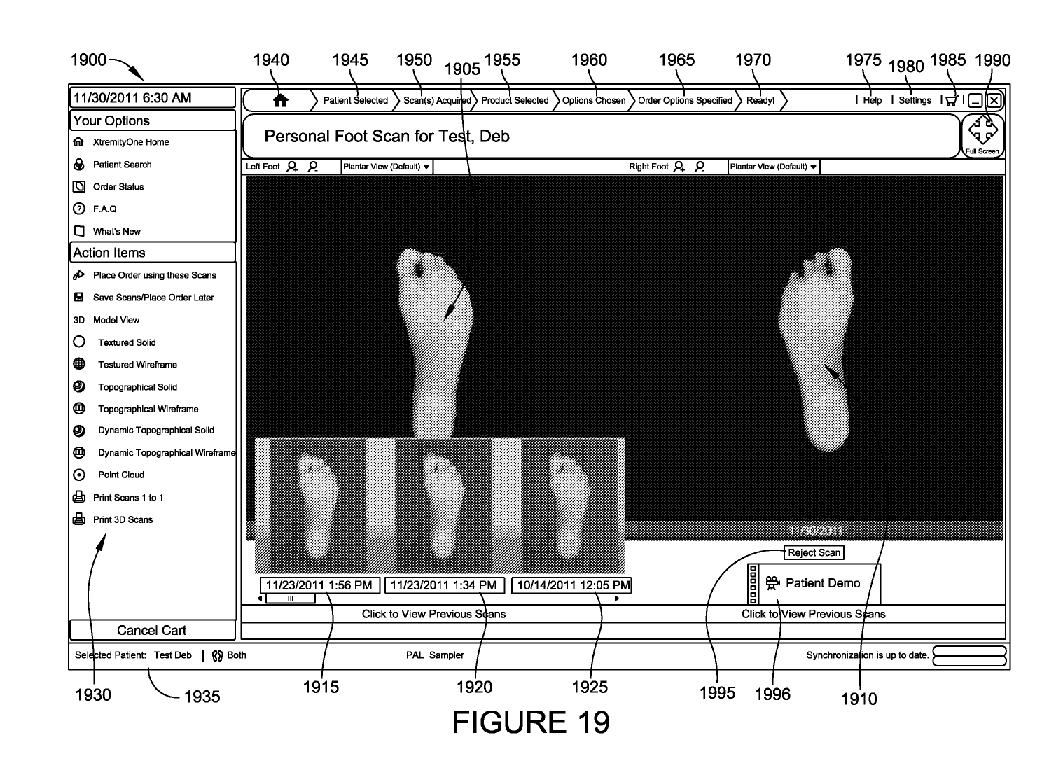

FIG. 19 illustrates a GUI 1900 of a management system for patient scans 1905, 1910, 1915, 1920, and 1925 of various planar view scans of the left foot and the right foot. A toolbar 1930 may provide various features, including action items, for management and analysis of the foot scans. Various buttons may be provided in GUI 1900, and may include, but are not limited to, a home button 1940, a patient selected button 1945, a scan acquired button 1950, a product selected button 1955, a product options chosen button 1960, an order options specified button 1965, a ready button 1970, a help button 1975, a settings button 1980, and a cart button 1985. GUI 1900 may provide a full-screen button 1990. GUI 1900 may provide a reject scan button 1995. GUI 1900 may provide a patient demo video button 1996.

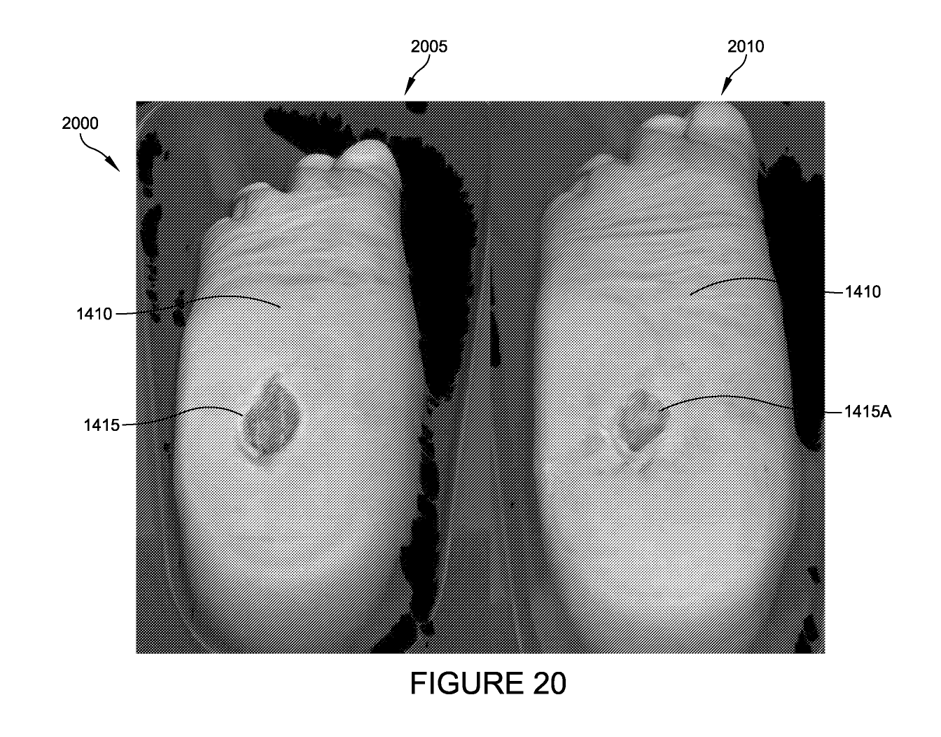

FIG. 20 is a progression 2000 illustrating two side-by-side images 2005 and 2010 showing a healing progression of a patient's wound 1415 and partially healed wound 1415A on the planar surface of the foot 1410.

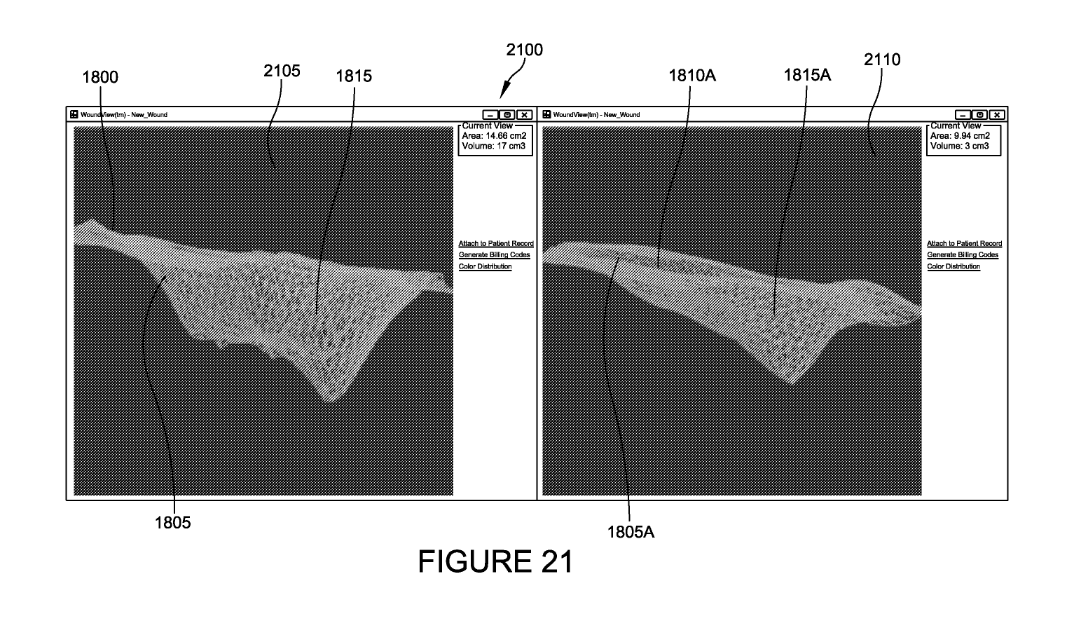

FIG. 21 illustrates a GUI 2105 in which there are shown wire mesh images 2105 and 2110 of the wounds 1810 and 1810A shown in FIG. 19.

All documents referenced herein are hereby incorporated by reference.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. A system for managing diabetic ulcers on a foot, comprising:
a scanner configured to obtain a two-dimensional image of a plantar surface of a foot;
an ulcer wound tracer tool configured to enclose a diabetic ulcer wound illustrated in the two-dimensional image separate from another portion of the surface of the foot;
an image processor configured to convert the two-dimensional image of the diabetic ulcer wound enclosed by the wound marking tool separate from the another portion of the surface of the foot via software into a three-dimensional map; and
a measurement calculator, using the three-dimensional map, configured to determine at least one of a surface area of the diabetic ulcer wound along the surface of the foot and a volume of the wound below the surface of the foot.

2. The system of claim 1, wherein the ulcer wound tracer tool provides a polygonal shape selectable to conform to a shape of the diabetic ulcer wound.

3. The system of claim 1, wherein the polygonal shape is a rectangle.

4. The system of claim 1, wherein the ulcer wound tracer tool provides a freehand tool selectable to confirm to edges of the diabetic ulcer wound.

5. The system of claim 1, further comprising a storage medium to retain multiple images, obtained on different dates from one another, of the two-dimensional image of the foot.

6. The system of claim 5, further comprising a graphical user interface configured to display at least two of the multiple images, obtained on different dates from one another, of the two-dimensional image of the foot.

7. The system of claim 1, further comprising a storage medium to retain multiple images, obtained on different dates from one another, of the three-dimensional map of the foot.

8. The system of claim 7, further comprising a graphical user interface configured to display at least two of the multiple images, obtained on different dates from one another, of the three-dimensional map of the foot.

9. The system of claim 7, further comprising a storage medium to retain multiple images, obtained on different dates from one another, of the surface area determined by the measurement calculator for the surface area of the diabetic ulcer wound along the surface of the foot.

10. The system of claim 7, further comprising a storage medium to retain multiple images, obtained on different dates from one another, of the volume determined by the measurement calculator for the volume of the wound below the surface of the foot.

11. A method of wound care management for a foot, comprising:
scanning, with a scanner, a two-dimensional image of the surface of a foot;
marking, using an ulcer wound tracer tool, to enclose an ulcer wound illustrated in the two-dimensional image separate from another portion of the surface of the foot;
converting, using an image processor, the two-dimensional image of the ulcer wound enclosed by the ulcer wound tracer tool separate from the another portion of the surface of the foot into a three-dimensional map; and
measuring, using the three-dimensional map, to determine at least one of a surface area of the diabetic ulcer wound along the surface of the foot and a volume of the diabetic ulcer wound below the surface of the foot.

12. The method of claim 11, wherein the ulcer wound tracer tool provides a polygonal shape selectable to conform to the wound.

13. The method of claim 11, wherein the polygonal shape is a rectangle.

14. The method of claim 11, wherein the ulcer wound tracer tool provides a freehand process selectable to conform to edges of the wound.

15. The method of claim 11, further comprising a step of providing a storage medium to retain multiple images, obtained on different dates from one another, of the two-dimensional image of the foot.

16. The method of claim 15, further comprising a step of providing a graphical user interface configured to display at least two of the multiple images, obtained on different dates from one another, of the two-dimensional image of the foot.

17. The method of claim 11, further comprising a step of providing a storage medium to retain multiple images, obtained on different dates from one another, of the three-dimensional map of the foot.

18. The method of claim 17, further comprising a step of providing a graphical user interface configured to display at least two of the multiple images, obtained on different dates from one another, of the two-dimensional image of the foot.

19. The method of claim 11, further comprising a step of providing a storage medium to retain multiple images, obtained on different dates from one another, of the surface area determined by the measurement calculator for the surface area of the wound along the surface of the foot.

20. The method of claim 11, further comprising a step of providing a storage medium to retain multiple images, obtained on different dates from one another, of the volume determined by the measurement calculator for the volume of the wound below the surface of the foot.

21. A system for managing diabetic ulcers on a foot, comprising:
an imaging device configured to obtain a two-dimensional image of a plantar surface of a foot;
an image processor configured to convert at least a portion of the two-dimensional image of the plantar surface of the foot having a diabetic ulcer wound into a wireframe three-dimensional map; and
a measurement calculator, using the wireframe three-dimensional map, configured to calculate at least one of a surface area of the diabetic ulcer wound along the surface of the foot and a volume of the wound below the surface of the foot.

22. The system of claim 21, further comprising a storage medium to retain multiple images, obtained on different dates from one another, of the two-dimensional image of the foot.

23. The system of claim 22, further comprising a graphical user interface configured to display at least two of the multiple images, obtained on different dates from one another, of the two-dimensional image of the foot.

24. The system of claim 21, further comprising a storage medium to retain multiple images, obtained on different dates from one another, of the wireframe three-dimensional map of the foot.

25. The system of claim 24, further comprising a graphical user interface configured to display at least two of the multiple images, obtained on different dates from one another, of the wireframe three-dimensional map of the foot.

26. The system of claim 24, further comprising a storage medium to retain multiple images, obtained on different dates from one another, of the surface area determined by the measurement calculator for the surface area of the diabetic ulcer wound along the surface of the foot.

27. The system of claim 24, further comprising a storage medium to retain multiple images, obtained on different dates from one another, of the volume determined by the measurement calculator for the volume of the wound below the surface of the foot.

28. A method of wound care management for a foot, comprising:
obtaining, with an imaging device, a two-dimensional image of the surface of a foot;
processing, using an image processor, the two-dimensional image of the two-dimensional image having a diabetic ulcer wound into a wireframe three-dimensional map; and
measuring, using the wireframe three-dimensional map, to calculate at least one of a surface area of the diabetic ulcer wound along the surface of the foot and a volume of the diabetic ulcer wound below the surface of the foot.

29. The method of claim 28, further comprising a step of providing a storage medium to retain multiple images, obtained on different dates from one another, of the two-dimensional image of the foot.

30. The method of claim 29, further comprising a step of providing a graphical user interface configured to display at least two of the multiple images, obtained on different dates from one another, of the two-dimensional image of the foot.

31. The method of claim 28, further comprising a step of providing a storage medium to retain multiple images, obtained on different dates from one another, of the three-dimensional wireframe map of the foot.

32. The method of claim 31, further comprising a step of providing a graphical user interface configured to display at least two of the multiple images, obtained on different dates from one another, of the three-dimensional wireframe map of the foot.

33. The method of claim 28, further comprising a step of providing a storage medium to retain multiple images, obtained on different dates from one another, of the surface area determined by the measurement calculator for the surface area of the wound along the surface of the foot.

34. The method of claim 28, further comprising a step of providing a storage medium to retain multiple images, obtained on different dates from one another, of the volume determined by the measurement calculator for the volume of the wound below the surface of the foot.
